(19) United States
(12) Patent Application Publication
(10) Pub. No.: US 2015/0079156 A1
(43) Pub. Date: Mar. 19, 2015

(54) LIPOSOMAL DELIVERY SYSTEM

(71) Applicant: THE QUEEN'S UNIVERSITY OF BELFAST, Belfast (GB)

(72) Inventors: Victoria Kett, Belfast (GB); Helmy Yusuf, Belfast (GB); Helen McCarthy, Belfast (GB); Kun Hung Chen, Hsinchu city (TW)

(21) Appl. No.: 14/385,367

(22) PCT Filed: Mar. 15, 2013
(86) PCT No.: PCT/EP2013/055471

§ 371 Date: Sep. 15, 2014

(30) Foreign Application Priority Data
Mar. 16, 2012 (GB) 1204632.2

(51) Int. Cl. A61K 9/127; A61K 9/00; A61K 38/38

(52) U.S. Cl. CPC A61K 9/1272; A61K 38/385; A61K 9/0043
USPC 424/450; 514/15.2

(57) ABSTRACT

The present invention is directed to a liposomal delivery system comprising defined lipid components, including dimethyldioctadecylammonium and other lipid components and a stabilizing agent, which may be used in the delivery of bioactive agents to a subject in need thereof. The liposomal delivery system of the invention provide improvements in terms of delivery and stability. Ideally, the liposomal delivery system is used as a vaccine or drug/medicament delivery system although other bioactive agents may be delivered.

### INTRODUCTION

The present invention is directed to a liposomal delivery system for the delivery of bioactive agents. Ideally, the liposomal delivery system is used as a vaccine or drug/medicament delivery system, although other bioactive agents may be contemplated.

The delivery of drugs or other macromolecular agents to the human or animal body, for example to a particular site, at a particular concentration or at a particular time, is a key requirement of modern medicine. Thus, there is a continual need for the development of new drug delivery systems which allow targeted delivery and overcome any natural barries within the human or animal body. For example, most drugs or medicaments exhibit poor penetration and/or stability when administered alone via a mucosal route. Thus, there is a need to develop pharmaceutical formulations that are able to deliver a therapeutic payload mucosally. Many solutions have been put forward. For example, encapsulating a drug into a vehicle enables improved stability if formulated appropriately. Liposomal delivery agents have been devised to achieve this aim.

The present invention is directed to an improved liposomal delivery system.

According to a first general aspect of the invention, there is provided a liposomal delivery system comprising or consisting of
a minor lipid component dimethyldioctadecylammonium (DDA);
a major lipid component or components, preferably selected from one or more of the following glycerolipid, glycerophospholipid, and/or sphingolipid or a combination thereof, which is capable of forming a stable lipid bilayer with the minor lipid component dimethyldioctadecylammonium (DDA); and
a tocopherol polyethylene glycol (PEG) ester derivative;
wherein the liposomal delivery system does not comprise the lipid cholesterol.

According to a second general aspect of the invention, there is provided a liposomal delivery system comprising or consisting of
less than approximately 50% by weight based on the total weight of the system, of a minor lipid component dimethyldioctadecylammonium (DDA);
more than approximately 50% by weight based on the total weight of the system, of a major lipid component or components selected from one or more of the following glycerolipid, glycerophospholipid, and/or sphingolipid or a combination thereof which is capable of forming a stable lipid bilayer with the minor lipid component dimethyldioctadecylammonium (DDA); and
less than approximately 10%, by weight based on the total weight of the system, of a tocopherol polyethylene glycol (PEG) ester derivative.

According to the first and second general aspects of the invention, there is provided a liposomal delivery system comprising or consisting of less than approximately 50%, preferably less than or equal to approximately 45%, 40%, 35%, 30%, 25% or 20%, by weight based on the total weight of the system of a minor lipid component dimethyldioctadecylammonium (DDA); less than or equal to approximately 9%, 8%, 7.7%, 7%, 6%, 5%, 4.5%, 4.3%, 4.2%, 4.1%, 4%, 3.9%, 3.8%. 3.7%, 3.6%, 3.5%, 3.4%, 3.3%, 3.2%, 3.1%, 3%, 2.5%, 2%, 1.5%, 1% or 0.5% of a tocopherol polyethylene glycol (PEG) ester derivative; and more than approximately 50%, preferably approximately 55%, 60%, 65%, 70%, 75%, 80% or more, by weight based on the total weight of the system, of a major lipid component or components which is capable of forming a stable lipid bilayer with dimethyldioctadecylammonium (DDA). It will be understood that this liposomal delivery system does not comprise the lipid cholesterol.

According to a third general aspect of the invention, there is provided a bioactive agent liposomal delivery complex comprising the liposomal delivery system of the invention and a bioactive agent.

According to a fourth general aspect of the invention, there is provided a pharmaceutical composition comprising the liposomal delivery system of the invention, a bioactive agent and a pharmaceutically acceptable excipient.

According to a fifth general aspect of the invention, there is provided the liposomal delivery system, bioactive agent liposomal delivery complex or the pharmaceutical composition of the invention for use in therapy.

According to a sixth general aspect of the invention, there is provided a method for the delivery of a bioactive agent to a subject using the liposomal delivery system, bioactive agent liposomal delivery complex or the pharmaceutical composition of the invention wherein the bioactive agent or agent is adsorbed to or encapsulated within the liposomal delivery system to form a bioactive agent liposomal delivery complex and the method comprises adminstering the bioactive agent liposomal delivery complex or the pharmaceutical composition to a subject in need thereof.

In this specification, it will be understood that the terms “liposomal delivery system”, “liposome” and “liposome formulation” are interchangable. The term “liposome” also covers “liposome particle”, “discrete liposome particles” and “discrete liposomes”. The liposomal delivery system of the invention does not include the bioactive agent. When the bioactive agent is added to the liposomal delivery system of the invention it is known as the bioactive agent liposomal delivery complex.

In this specification, it will be understood that the terms “bioactive agents” and “biologically active agents” are interchangeable and cover a range of therapeutic and non-therapeutic agents.

In this specification, except where indicated, it will be understood that the percentages described in relation to the liposomal delivery system are relative to the sum of the dry weight of each of the components added and excluding any bioactive agent. In some embodiments of the invention, the percentages described in relation to the liposomal delivery system are relative to the sum of the dry weight of each of the components added to give the liposomal delivery system 100% by mass and excluding any bioactive agent.

In this specification, it will be understood that the term “TPGS” refers to “D-alpha tocopherol polyethylene glycol succinate corresponding to “2-Hydroxyethyl 2,5,7,8-tetramethyl-2-(4,8,12-trimethyltridecyl)-3,4-dihydro-2H-chromen-6-yl succinate”.

In this specification, it will be understood that the abbreviations DDA refers to didecyldimethylammonium and covers dimethyldioctadecylammonium-bromide, dimethyldioctadecylammonium-chloride, dimethyldioctadecylammonium-phosphate and/or dimethyldioctadecylammonium-acetate. For example, DDAB correspond to Didecyldimethylammonium bromide. It will be understood that didecyldimethylammonium bromide, N-Decyl-N,N-dimethyldecan-1-aminium bromide, 1-decanaminium, N-decyl-N,N-dimethyl-, bromide (1:1), N-Decyl-N,N-dimethyldecan-1-aminium bromide, didecyldimethylamine, bromide, Didecyldimethylammonium Bromide and Didecyldimethylammoniumbromide refer to the same lipid component. Similarly, the bromide portion of DDAB may be replaced with chloride, phosphate or acetate.

In this specification, it will be understood that phosphatidylcholine (PC) is commonly used to refer to a range of lipids with the same basic structure known as glycerophosphocholines (as defined on the LIPID MAPS database), which may also be referred to as lecithin. PC from different sources will contain slightly different proportions and/or combinations of phosphatidyl cholines. For example, PC is also available from plant and animal sources that include but are not limited to egg, human plasma and erythrocytes, rat lung, heart liver, bovine sources including brain, plants such as Arabidosis thaliana and yeasts such as Lipomyces lipoferus

In this specification, it will be understood that the phosphatidylcholine may be selected from soy phosphatidylcholine (SPC), egg phosphatidylcholine or from any other available natural or non-natural/synthetic sources.

In this specification, it will be understood that the terms lyophilization and freeze-drying refer to the same drying process and may be used interchangeably.

According to a first general aspect, the present invention is directed to a liposomal delivery system comprising discrete liposomes, the system comprising or consisting of
a minor lipid component dimethyldioctadecylammonium (DDA);
a major lipid component or components selected from one or more of the following glycerolipid, glycerophospholipid, and/or sphingolipid or a combination thereof which is capable of forming a stable lipid bilayer with the minor lipid component dimethyldioctadecylammonium (DDA); and
a tocopherol polyethylene glycol (PEG) ester derivative;
wherein the liposomal delivery system does not comprise the lipid cholesterol.

In this first general aspect, it will be understood that the amount (e.g. percentage by weight, molar ratio etc) of the major lipid component present in the liposomal delivery complex is greater than the amount (e.g. percentage by weight, molar ratio etc) of the minor lipid component present in the liposomal delivery complex.

We postulate that liposomes based on tocopherol polyethylene glycol (PEG) ester derivative:minor lipid component DDA:major lipid component (such as TPGS:DDA:PC) are stable when the amount of major lipid component>DDA>tocopherol polyethylene glycol (PEG) ester derivative (such as SPC>DDA>TPGS).

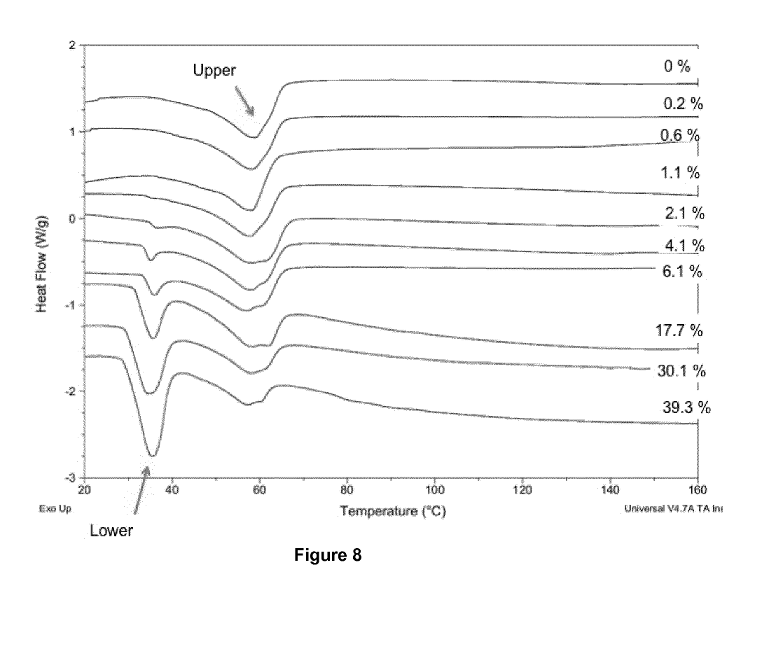

Ideally, the major lipid component (e.g. PC/SPC) contribution should be greater than 50% by weight of the composition and preferably greater than or equal to 80% by weight (see Tables 1, 4 and 5). Ideally, the DDA concentration should be less than 50% by weight of the composition and preferably less than or equal to 20% by weight (see Tables 1, 4 and 5). The tocopherol polyethylene glycol (PEG) ester derivative (i.e. TPGS) concentration should be less than 10% by weight and preferably less than 7.7%, (see Table 4 and FIG. 8), more preferably 4.1% or less as shown in FIG. 8, even more preferably the tocopherol polyethylene glycol (PEG) ester derivative (i.e. TPGS) concentration should be 1% by weight or less as indicated by phase separation for the “Lower” Tm as seen in FIG. 8.

According to a second general aspect, the present invention is directed to a liposomal delivery system comprising or consisting of less than approximately 50%, preferably less than or equal to approximately 45%, 40%, 35%, 30%, 25% or 20%, by weight based on the total weight of the system of a minor lipid component dimethyldioctadecylammonium (DDA); less than approximately 10%, 9%, 8%, 7.7%, 6%, 5%, 4.5%, 4.3%, 4.2%, 4.1%, 4%, 3.9%, 3.8%. 3.7%, 3.6%, 3.5%, 3.4%, 3.3%, 3.2%, 3.1%, 3%, 2.5%, 2%, 1.5% or 1% by weight of a tocopherol polyethylene glycol (PEG) ester derivative; and more than approximately 50%, preferably approximately 55%, 60%, 65%, 70%, 75%, 80% or more, by weight based on the total weight of the system, of a major lipid component or components which is capable of forming a stable lipid bilayer with dimethyldioctadecylammonium (DDA).

In this manner, in all aspects and embodiments of the invention, the minor lipid component dimethyldioctadecylammonium (DDA) may be present less than approximately 15%, 10% or 5% by weight based on the total weight of the system and the major lipid component or components which is/are capable of forming a stable lipid bilayer with dimethyldioctadecylammonium (DDA) may be present from approximately 85%, 90% or 95% by weight based on the total weight of the system.

According to a preferred embodiment, the liposomal delivery system of the invention comprises or consists of the minor lipid component dimethyldioctadecylammonium (DDA) which may be present from approximately 15% to 20% by weight based on the total weight of the system; the major lipid component or components, which is/are capable of forming a stable lipid bilayer with dimethyldioctadecylammonium (DDA), and may be present from approximately 80% to 85% by weight based on the total weight of the system; and the tocopherol polyethylene glycol (PEG) ester derivative which may be present from approximately 0.01% to 10% by weight based on the total weight of the system. The greater the amount of the minor lipid component DDA, the less tocopherol polyethylene glycol (PEG) ester derivative included in the system.

In all aspects and embodiments of the invention, the liposomal delivery system must comprise three essential components, the major lipid component, the minor lipid component dimethyldioctadecylammonium (DDA) and the tocopherol polyethylene glycol (PEG) ester derivative. It will be understood that the percentages by weight of each of these essential components is by reference to the total weight of the system and correlates to the amount of each component used in the manufacture of the liposomal delivery system. In a most preferred embodiment the percentages of each of these three components adds up to 100 percent. Thus, it will be understood that each of the three essential components must be present above 0.01% by weight based on the weight of the system.

As defined above, the major lipid component or components is selected from one or more of the following glycerolipid, glycerophospholipid and/or sphingolipid or a combination thereof (as defined in Fahy, E, Subramaniam, S, Murphy, R C, Nishijima, M, Raetz, C R H, Shimizu, T, Spener, F, van Meer, G, Wakelam, M J O, and Dennis, E A Update of the LIPID MAPS comprehensive classification system for lipids. Journal of Lipid Research 2009 April; 50(Supplement): S9-S14).

Preferably, the first essential component, the major lipid component or components is selected from one or more of the following diacylglycerophosphocholines, glycerophosphates, glycerophosphoethanolamines, glycerophosphoglycerols, glycerophosphoglycero-phosphoglycerols and/or glycerophosphoserines.

The second essential component, the lipid DDA is known to stimulate an immune response when used to deliver antigen (Holten-Andersen L, Doherty T M, Korsholm K S, Andersen P. Combination of the cationic surfactant dimethyl dioctadecyl ammonium bromide and synthetic mycobacterial cord factor as an efficient adjuvant for tuberculosis subunit vaccines. Infection and Immunity 2004 March; 72(3):1608-1; Brandt L, Elhay M, Rosenkrands I, Lindblad E B, Andersen P. ESAT-6 subunit vaccination against Mycobacterium tuberculosis. Infection and Immunity 2000 February; 68(2):791-5). However, DDA liposomes have some drawbacks including physical instability, as they easily aggregate in the presence of small amounts of salt or even in pure water (Mohammed A R, Bramwell V W, Coombes A G A, Perrie Y. Lyophilisation and sterilisation of liposomal vaccines to produce stable and sterile products. Methods 2006 September; 40(1):30-8). Thus, due to this disadvantages the use of DDA in a liposome formulation is unexpected.

The third essential component is a tocopherol polyethylene glycol (PEG) ester derivative. Ideally, tocopherol polyethylene glycol (PEG) is used. For example, D-alpha tocopherol polyethylene glycol 1000 succinate (TPGS) may be used. TPGS is known to increase liposome stability and to enhance cell uptake. The amphiphile structure of D-alpha tocopherol polyethylene glycol 1000 succinate (TPGS), a preferred tocopherol polyethylene glycol (PEG) ester derivative, comprises a PEG portion as the polar head and tocopherol succinate as the lipophilic tail. It has been shown that α-tocopherol has an ordering effect on the fatty acyl tails of lipid membranes in the liquid crystalline state because the insertion of α-tocopherol can restrict the space of the fatty acids which acts to stabilize these membranes (Hincha D K. Effects of alpha-tocopherol (vitamin E) on the stability and lipid dynamics of model membranes mimicking the lipid composition of plant chloroplast membranes. Febs Letters 2008 Oct. 29; 582(25-26):3687-92]. In addition, it was reported that incorporation of TPGS in microparticles increases the immune response towards diphtheria toxoid (Somavarapul S, Pandit S, Gradassi G, Bandera A, Ravichandran E, Alpar O H. Effect of vitamin E TPGS on immune response to nasally delivered diphtheria toxoid loaded poly(caprolactone) microparticles. International Journal of Pharmaceutics 2005 Jul. 25; 298(2):344-7.).

However, the use of this combination of a tocopherol PEG ester derviative, in particular TPGS, and DDA, without cholesterol, in a liposomal delivery system has not been contemplated previously. Unexpectedly, we have found that this combination of DDA (the “minor lipid component”) and a tocopherol PEG ester derviative, such as TGPS, overcomes the problems associated with DDA outlined above.

Furthermore, we have surprisingly found that the liposomal delivery system of the invention does not require the use of cholesterol which is used in most commercially available liposomes containing DDA as a membrane stabilizing agent. Cholesterol tends to oxidise, potentially leading to the build-up of toxic components in cholesterol-containing formulations. It is also generally sourced from animal material with the potential for viral contamination. Therefore, formulating DDA liposomes without cholesterol is advantageous. Although, a third lipid component is present in the liposomal delivery system of the invention, the “major lipid component”, this third lipid component does not include cholesterol. In addition, the major lipid component does not include DDA (the “minor lipid component”). Thus, the third lipid component is selected from one or more of the following glycerolipid, glycerophospholipid and/or sphingolipid. This is a significant advantage of the invention.

Thus, we have unexpectedly found that the inclusion of a tocopherol PEG ester derviative, such as TPGS, removes the need for cholesterol in the liposomal delivery system of the invention. We have found that this liposomal delivery system of the invention is stable without the inclusion of cholesterol. This is unexpected as most commercially available liposomes containing DDA utilise cholesterol to maintain rigidity and prevent fusion between lipid bilayers.

We have also found that the use of DDA and a tocopherol PEG ester derviative, such as TPGS, in the presence of a third lipid component (the “major lipid component”) renders the liposomes stable to the subsequent drying process. This is a significant manufacturing processing benefit and post-manufacturing adminstration benefit.

Additionally, we have also found that the liposomal delivery system of the invention offers both improved mucus penetrating ability and the ability to improve cell penetration/cell uptake properties once across the mucosal barrier.

Hence, the liposomal delivery system of the invention is an improved system over known liposomes.

We have characterised liposomes according to the invention which contain a tocopherol PEG ester derviative, such as TPGS, and DDA loaded with a sample antigen and have found that
the liposomes are in the correct size range for mucosal penetration. We have found that each discrete liposome within the liposomal delivery system has an average diameter less than 150 nm. The liposome size range of the liposome delivery system with a bioactive agent is typically in the range from 100 nm to 300 nm. For example, we have found typical size ranges of 200-300 nm when the liposome delivery system is loaded with a model antigen;
nasal tissue penetration studies have shown that the liposomes cross the nasal mucosa and exhibit penetration to the basal layer; and
the liposomes are able to penetrate lung epithelial cells and have no deleterious effect on cell viability.

We have also advantageously found that liposomes containing a tocopherol PEG ester derviative, such as TPGS, and DDA exhibit superior performance in terms of nasal tissue and lung epithelial cell pentration compared to liposomes without a tocopherol PEG ester derviative, such as TPGS.

It will also be understood that the major lipid component or components comprises lipids, excluding cholesterol, selected from one or more of the following diacylglycerophosphocholines, glycerophosphates, glycerophosphoethanolamines, glycerophosphoglycerols, glycerophosphoglycero-phosphoglycerols and/or glycerophosphoserines, which are capable of forming a stable lipid bilayer with the minor lipid component dimethyldioctadecylammonium (DDA).

Accordingly, the major lipid component or components may be selected from one or more of the following diacylglycerophosphocholines, glycerophosphates glycerophosphoethanolamines, glycerophosphoglycerols, glycerophosphoglycero-phosphoglycerols and/or glycerophosphoserines or a combination thereof. Specific lipids that may also be used in accordance with the invention include phosphatidylcholine (PC), lecithin, oleic acid, sorbitan trioleate, sorbitan mono-oleate, sorbitan monolaurate, stearylamine, N-[1-(2,3-Dioleoyloxy)propyl]-N,N,N-trimethylammonium methyl-sulfate (DOTAP), ceramide, ceramide carbamoyl-spermine (CCS), dicetylphosphate (DCP), 1,2-dilinoleoyl-sn-glycero-3-phosphocholine (DLiPC), 1,2-dilauryl-sn-glycero-3-phosphocholine (DLPC); 1,2-dimyristoyl-sn-glycero-3-phosphocholine (DMPC); 1,2-dipalmitoyl-sn-glycero-3-phosphoethanolamine-N-[6-[(2,4-dinitrophenyl)amino]hexanoyl], 1,2-dioleoyl-sn-glycero-3-phosphocholine (DOPC); 1,2-dioleoyl-3-trimethylammonium propane (DOTAP); 1-[2-(oleoyloxy)ethyl]-2-oleyl-3-(2-hydroxyethyl)imidazolinium (DOTIM), 1,2-dipalmitoyl-sn-glycero-3-phosphoethanolamine (DPPC), 1,2-dipalmitoyl-sn-glycero-3-phosphoethanolamine; (DPPE), 1,2-dipalmitoyl-sn-glycero-3-phosphoethanolamine (DPyPE), 1,2-diphytanoyl-sn-glycero-3-phosphoethanolamine; 1,2-distearoyl-sn-glycero-3-phosphocholine (DSPC) 1,2-distearoyl-sn-glycero-3-phosphoethanolamine (DSPE), 1,2-diacyl-sn-glycero-3-phosphoglycerol (PG), N-(3-aminopropyl)-N,N-dimethyl-2,3-bis(cis-9-tetradeceneyloxy)-1-propanaminium (GAP-DMORIE), glyceryl monostearate (GMS), lysophosphatidylcholine (LPC), 2-monopalmitoylglycerol (MPG), monophosphoryl lipid A (MPL); octadecanoic acid, palmitic acid; PC, 1,2-diacyl-sn-glycero-3-phosphocholine, 1,2-diacyl-sn-glycero-3-phosphoglycerol (PG), 1-palmitoyl-2-linoleoyl-sn-glycero-3-phosphocholine (PLiPC), 1,2-diacyl-sn-glycero-3-phosphoserine (PS), sorbitan monostearate (SMS), sphingosine-1-phosphate (SP1), (N-succinimidyl-3-(2-pyridyldithio)-propionate) (SPDP), and/or α,α,-trehalose-6,6′-dibehenate (TDB) or their derivatives. Natural or synthetic lipids may be contemplated.

According to a most preferred embodiment of the invention, ideally the major lipid component is a phosphatidylcholine (PC).

According to another preferred embodiment of the invention, the tocopherol PEG ester derivative is present less than 9%, 8%, 7.7%, 7%, 6%, 5%, 4.5%, 4.3%, 4.2%, 4.1%, 4%, 3.9%, 3.8%. 3.7%, 3.6%, 3.5%, 3.4%, 3.3%, 3.2%, 3.1%, 3%, 2.5%, 2%, 1.5% 1.0% or 0.5%, preferably from 0.5% to 2%, more preferably less than 1%, even more preferably less than 0.5% by weight based on the total weight of the system. The tocopherol PEG ester derivative is an essential component of the invention and must be present above 0.01% by weight based on the total weight of the system.

Preferably, the tocopherol PEG derivative comprises a PEG portion which is less than 10 KDa, preferably from 1 to 10 kDa, more preferably 1 to 5 kDa. This size limitation is important as there is evidence to suggest that PEG 10 kDa might be too long for efficient transport through mucus so a size less than 10 Kda is preferable for mucosal adminstration (Wang Y Y, Lai S K, Suk J S, Pace A, Cone R, Hanes J, Angew. Chem. Int. Ed 2008 47, 9726-9729).

The tocopherol PEG ester derivative acts as a surface-altering agent and is associated with the DDA liposome. We postulate that that the tocopherol PEG ester derivative may be incorporated into the lipid bilayer membrane or alternatively surface associated, embedded, enmeshed or coupled with the lipid bilayer membrane. Additionally, the tocopherol PEG ester derivative may be used to link or attach to other molecules or agents.

It will be understood that the tocopherol PEG ester derivative may be selected from one of more of the following tocopherol sebacate polyethylene glycol, tocopherol dodecanodioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol and/or tocopherol phthalate polyethylene glycol.

Alternatively, the tocopherol PEG ester derivative is a delta tocopherol or alpha tocopherol PEG derivative. For example, the PEG ester derivative may be D-alpha tocopherol polyethylene glycol 1000 succinate.

It will be understood that other PEG (polyethylene glycol) derivatives with similar characteristics and properties to TPGS may also be used. For example, similar surface agents or surfactants could be formed using delta tocopherol in place of alpha tocopherol. In addition, surface agents or surfactants with similar characteristics as TPGS could be made by bonding PEG to other hydrophobic or charged molecules including acetic acid, propionic acid or butyric acid. Alternatively, non-mucoadhesive polymers of appropriate molecular weight could also be used in place of PEG.

The minor lipid component is DDA. Although, it will be contemplated that lipids with similar characteristics and properties to dimethyldioctadecylammonium (DDA) may also be used.

According to a preferred embodiment, the minor lipid component dimethyldioctadecylammonium (DDA) is selected from one ore more of the following dimethyldioctadecylammonium-bromide, dimethyldioctadecylammonium-chloride, dimethyldioctadecylammonium-phosphate and/or dimethyldioctadecylammonium-acetate.

According to a more preferred embodiment, the minor lipid component is dimethyldioctadecylammonium-bromide (DDAB).

According to a preferred embodiment of the invention, the liposomal delivery comprises the minor lipid component dimethyldioctadecylammonium (DDA) and the PEG ester derivative D-alpha tocopherol polyethylene glycol succinate (TPGS).

According to yet another preferred embodiment of the invention, the liposomal delivery system comprises or consists of the major lipid component phosphatidylcholine, preferably soy phosphatidylcholine (SPC); the minor lipid component dimethyldioctadecylammonium (DDA); and the tocopherol PEG ester derivative D-alpha tocopherol polyethylene glycol succinate (TPGS).

In this embodiment, the liposomal delivery system comprising discrete liposomes, comprises or consists of
less than approximately 50% by weight based on the total weight of the system, of a minor lipid component dimethyldioctadecylammonium (DDA);
more than approximately 50% by weight based on the total weight of the system, of a major lipid component phosphatidylcholine which is capable of forming a stable lipid bilayer with the minor lipid component dimethyldioctadecylammonium (DDA); and
less than approximately 10%, 9%, 8%, 7.7%, 7%, 6%, 5%, 4.5%, 4.3%, 4.2%, 4.1%, 4%, 3.9%, 3.8%. 3.7%, 3.6%, 3.5%, 3.4%, 3.3%, 3.2%, 3.1%, 3%, 2.5%, 2%, 1.5%, 1% or 0.5% of a tocopherol polyethylene glycol (PEG) ester derivative.

According to an alternative embodiment, the liposomal delivery system comprises or consists of less than or equal to approximately 45%, 40%, 35%, 30%, 25% or 20% by weight, based on the total weight of the system, of a minor lipid component dimethyldioctadecylammonium (DDA); approximately 55%, 60%, 65%, 70%, 75%, 80% or more, by weight based on the total weight of the system, of a major lipid phosphatidylcholine; and less than approximately 10%, 9%, 8%, 7.7%, 7%, 6%, 5%, 4.5%, 4.3%, 4.2%, 4.1%, 4%, 3.9%, 3.8%. 3.7%, 3.6%, 3.5%, 3.4%, 3.3%, 3.2%, 3.1%, 3%, 2.5%, 2%, 1.5% 1.0% or 0.5%, preferably from 0.5% to 2%, more preferably less than 1%, even more preferably less than 0.5% of a tocopherol polyethylene glycol (PEG) ester derivative.

Typically, we have found that each discrete liposome, before addition of the bioactive agent, of the liposomal delivery system has an average diameter less than 150 nm. When a bioactive agent is mixed with or added to the liposome delivery system then the size range in terms of average diameter can increase to sizes in the typical range from 100 nm to 300 nm or even larger.

Ideally, the liposomal delivery system is cationic. However, the presence of the tocopherol PEG ester derivative has the effect of shielding the liposome surface to some extent. This can potentially prevent charge:charge interactions as might be expected to occur between the charged surface and the mucins in mucus Thus, the liposomal delivery system of the invention may be used to deliver postive and negatively charged active agents. This is another advantage of the invention.

The liposome delivery system of the invention is made according to standard practice and protocols. For example, the thin film hydration method may be used. Alternatively, other conventional methods may be used such as those discussed in Maherani, B, Arab-Tehrany, E, Mozafari, M R, Gaiani, C, Linder, M Liposomes: A Review of Manufacturing Techniques and Targeting Strategies. Current Nanoscience 2011 June; 7: 436-452.

The prepared liposomes may be sized by sonication, extrusion or according to other methods known in the art (Maherani, B, Arab-Tehrany, E, Mozafari, M R, Gaiani, C, Linder, M Liposomes: A Review of Manufacturing Techniques and Targeting Strategies. Current Nanoscience 2011 June; 7: 436-452).

The bioactive agent may be added to the liposomal delivery system of the invention by simple mixing or, for example, by the dehydration rehydration method. These methods are conventional manufacturing methods.

Once, the bioactive agent has been added to the liposomal delivery system to form the bioactive agent liposomal delivery complex, it may be freeze-dried to result in a solid or powder. The freeze-dried powder or solid requires may be adminstered as a powder or solid or can be rehydrated before use. We have advantageously found that the freeze-dried powders or solids of the invention are stable both before and after rehydration.

Alternatively, the liposomal delivery system may be freeze-dried and subsequently the bioactive agent may be introduced into the liposomes of the liposomal delivery system during a rehydration step at the point of administration to form the bioactive agent liposomal delivery complex.

The liposomal delivery system of the invention may be provided in any form. For example, the delivery system may be in the form of a fluid, dispersion, dried powder, solid, optionally produced by spray-drying, lyophilization (freeze drying) or spray freeze drying. The liposomal delivery system and the bioactive agent may be dried together or the bioactive agent may be introduced into the liposomes during a rehydration step at the point of administration.

We have advantageously found that the liposomal delivery system provides for a stable freeze dried solid or powder. We have advantageously found that the particular choice of components of the present invention provides for a stable dried solid or powder. We have advantageously found that inclusion of the bioactive agent with the liposomes before freeze drying obviates the need for separate mixing of a solution or dispersion of the bioactive agent with the freeze dried liposome delivery system at the point of administration in order to form the liposome bioactive agent complex.

The liposomal delivery system of the invention may be adapted for different delivery methods. For example, the liposomal delivery system of may be adapted for mucosal adminstration, preferably adapted for intranasal adminstration. For intranasal adminstration, the liposomal delivery system may be adapted for delivery as a solid, liquid, gel or powder that is preferentially deposited or adhered to the nasal mucosal surface. For pulmonary delivery the liposomal delivery system may be adapted for inhalation as a powder or liquid spray or mist. Other adminstration methods are contemplated, covering both local and systemic delivery means, such as injection, inhalation, oral, vaginal, rectal, buccal, intradermal, transdermal adminstration etc. that may require the use of specialist delivery devices including inhalers, creams, lotions, patches, gels, microneedles.

It will be understood that the liposomal delivery system of the invention further comprises a bioactive agent. The bioactive agents are adsorbed to or encapsulated within the liposomal delivery system to form a bioactive agent liposomal delivery complex. The amount and concentration of the bioactive agent will vary according to the therapeutic effect required and the route of delivery. The bioactive agent liposomal delivery complex may either be produced prior to freeze-drying or by reconstituting the freeze-dried liposomal delivery system with a fluid containing the bioactive agent.

According to another general aspect of the invention, there is provided a pharmaceutical composition comprising the liposomal delivery system of the invention, a bioactive agent and a conventional pharmaceutically acceptable excipient.

According to yet another general aspect of the invention, there is provided a liposomal delivery system, bioactive agent liposomal delivery complex or pharmaceutical composition of of the invention for use in therapy.

According to yet another general aspect of the invention, there is provided a method for the delivery of a bioactive agent to a subject using the liposomal delivery system according to the invention. As a first step the bioactive agent or agents are adsorbed to or encapsulated within the liposomal delivery system to form a bioactive agent liposomal delivery complex. This bioactive agent liposomal delivery complex is then delivered to a subject in need thereof.

In this manner the method for the delivery of a bioactive agent to a subject using the liposomal delivery system, bioactive agent liposomal delivery complex or the pharmaceutical composition of the invention comprises adminstering the liposomal delivery system, bioactive agent liposomal delivery complex or the pharmaceutical composition to a subject in need thereof.

It will be understood that the bioactive agent of the invention may be selected from one or more of the following, vaccines, immunogenic compositions, proteins, nucleic acids, drugs/medicaments, therapeutic agents, imaging agents and/or diagnostic agents.

Ideally, these liposomal delivery systems are used in the delivery of many different bioactive agents. These bioactive agents include, but are not limited to, vaccines or molecules which induce an immune response to bacterial or viral antigens, toxic proteins caused by disease, autoantigens or allergens. Advantageously, the liposome delivery system of the invention may be used in vaccine development.

Alternatively, drugs/medicaments, therapeutic agents, imaging or diagnostic agents may be adminstered. These bioactive agents are either adsorbed to or encapsulated within the liposomal delivery system to form a bioactive agent delivery complex/system.

In addition, the liposome delivery system may be used in the delivery of drugs or other therapeutic, imaging or diagnostic agent for local and/or systemic effect.

Any number of active agents may be adminstered. For example, insulin may be adminstered. Alternatively, cancer drugs/medicaments may be administered. Thus, the treatment of many different diseases or disorders may be contemplated.

According to one preferred embodiment of the invention, the liposomal delivery system may be formulated as dry powders and/or solids.

According to another preferred embodiment of the invention, the liposome delivery system of the invention, preferably TPGS-DDA based cationic liposomes, may be lyophilized for use as a delivery system.

According to another embodiment of the invention, the liposomal delivery system may be in liquid form and administered as a fluid or dispersion. For example by injection or oral adminstration.

Both fluids and powders or solids may be suitable for pulmonary, topical (including buccal or sublingual), gastrointestinal, ophthalmic, rectal or vaginal administration.

Advantageously, the liposomal delivery system may be adminstered mucosally.

According to one embodiment of the invention, the liposome may be adminstered intranasally, for example for inhalation.

Additionally, the liposomal delivery system may be administered by injection or other parenteral routes.

### DETAILED DESCRIPTION

The invention will now be described by the following non-limiting figures and examples.

In the following figures and examples “TPGS:DDA:SPC liposomes” refers to liposomes containing 1.0% TPGS, 16.6% DDA and 82.4% SPC (corresponding to Formulation 11 in Example 3). SPC liposomes refers to liposomes prepared in the same manner as the TPGS:DDA:SPC liposomes that contain SPC only. “DDA:SPC liposomes” refers to liposomes containing 16.8% DDA and 83.2% SPC by mass (corresponding to Formulation 3E in Example 3).

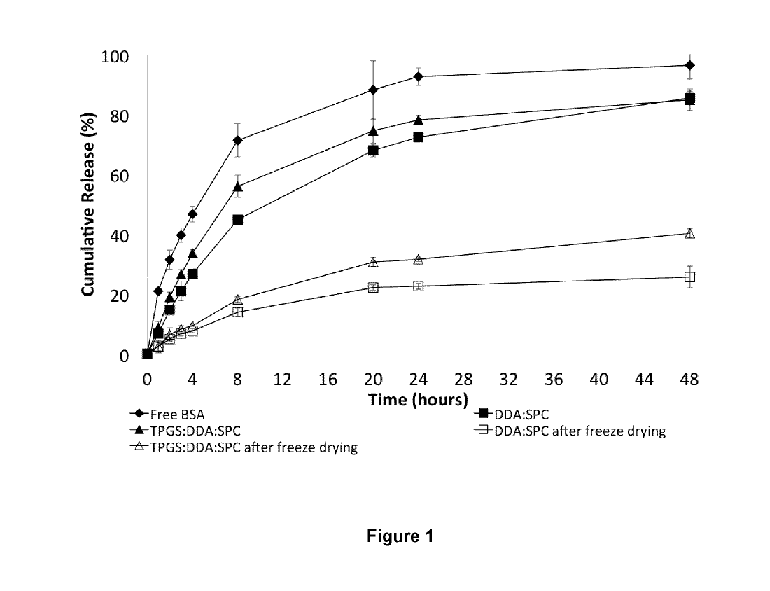

FIG. 1 shows the cumulative release of BSA from liposomes at 37° C. (n=3, ± represents SD).

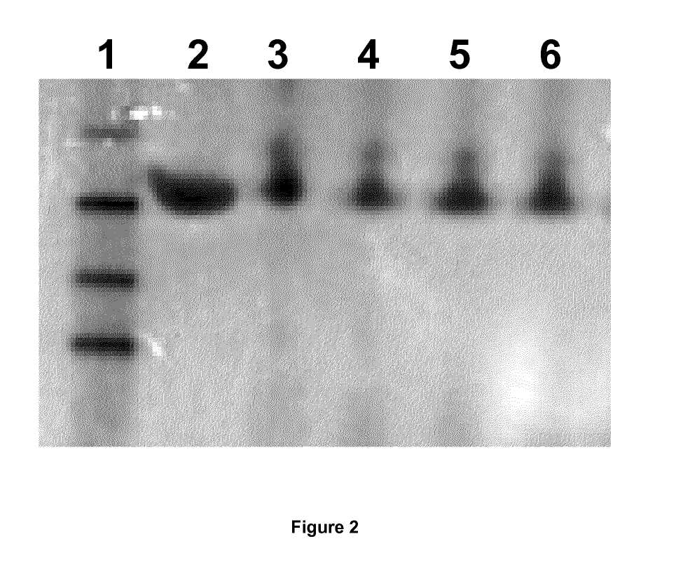

FIG. 2 shows the results from protein stability SDS-PAGE analysis. The different ladders of the gel consisted of the following composition: lane 1: protein markers; lane 2, native BSA; lane 3: BSA-loaded TPGS:DDA:SPC liposomes (before freeze drying) lane 4: BSA-loaded DDA:SPC Liposomes (before freeze drying) lane 5: BSA-loaded TPGS:DDA:SPC Liposomes (after freeze drying); lane 6: BSA-loaded DDA:SPC Liposomes (after freeze drying)

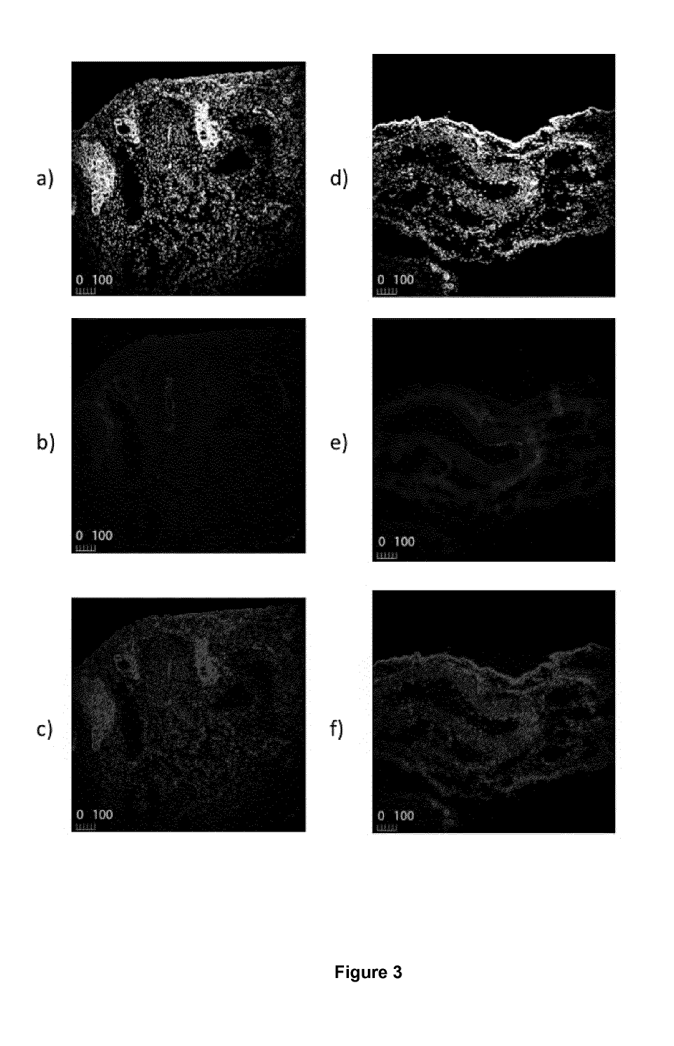

FIG. 3 shows CLSM images of cryosectioned bovine nasal tissues after 1 hour penetration of control (buffer) and reconstituted CF loaded liposomal dispersions under 60× magnification and dual staining with propidium iodide (PI). Images a) to c) control; d) to f) free CF; g) to i) SPC liposomes; j) to l) TPGS:DDA:SPC liposomes. Images a, d, g and j show PI staining. Images b, e, h and k show CF staining. Images c, f, i and l show dual staining.

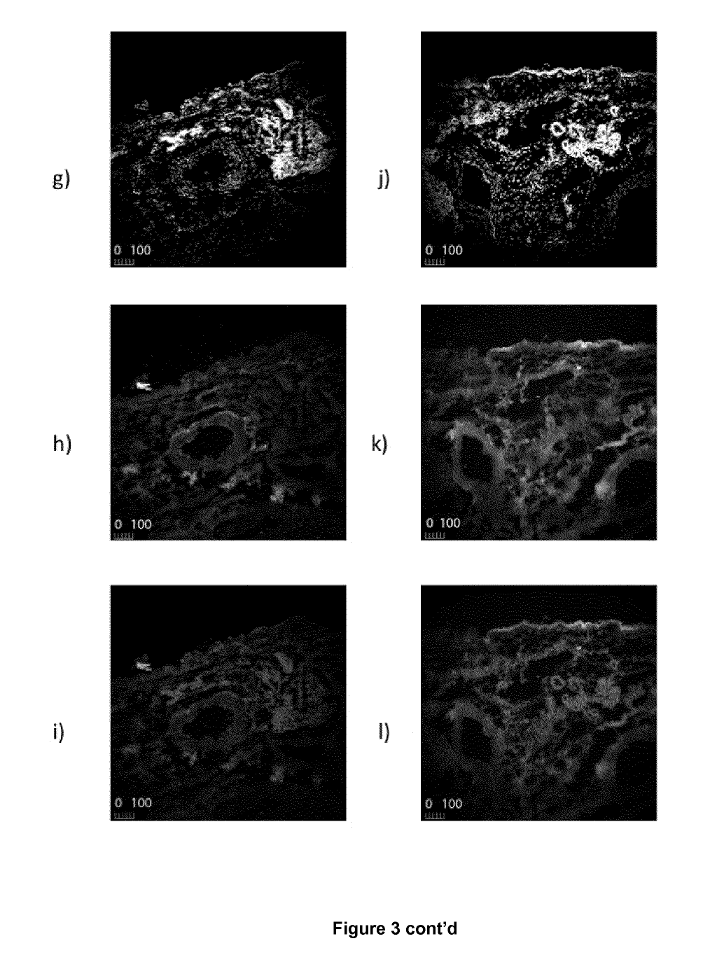

FIG. 4 shows cell flourescence intensity of human lung epithelilal cells incubated with CF-loaded liposomes at 37° C. Effect of liposomes entrapment on cellular uptake of CF. Results are mean value±SEM (n=3). ♦ refers to free CF, ▪ refers to SPC liposomes, Δ refers to TPGS:DDA:SPC liposomes.

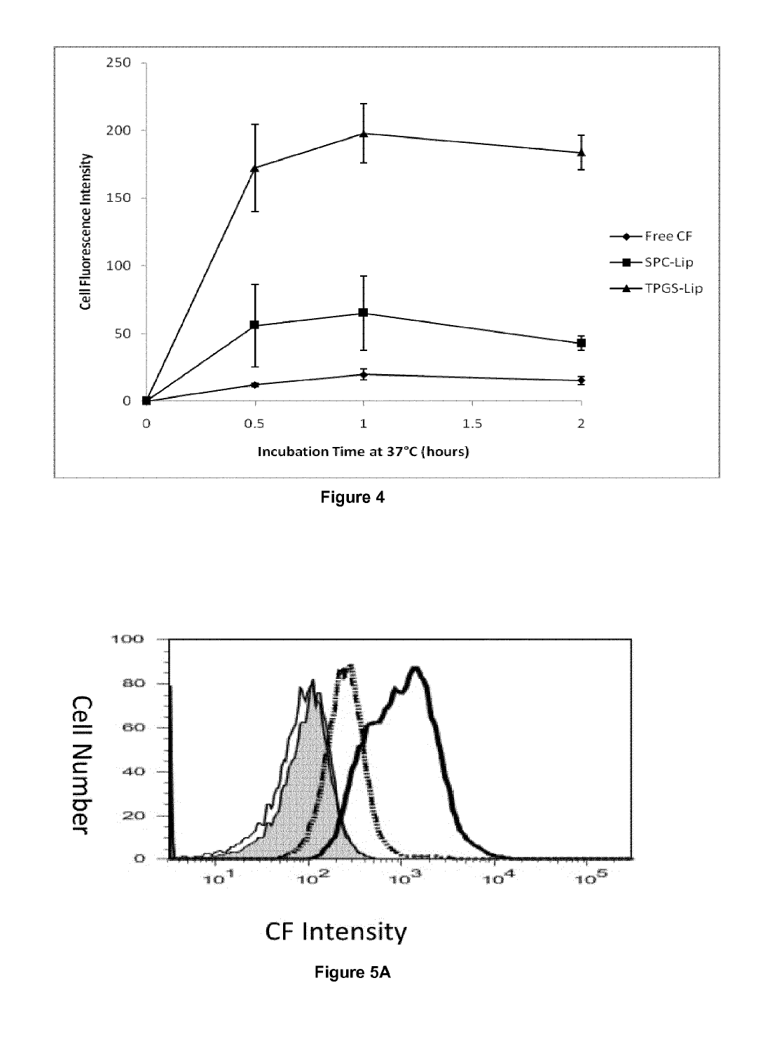

FIG. 5 shows flow cytometric analysis of cell-associated CF fluorescence after three different incubation times; (A) 0.5 h, (B) 1 h, (C) 2 h. Grey area (negative cells). thin solid line (blank lip), dashed line (free CF), grey line (CF-loaded SPC liposomes), black solid line (CF-loaded TPGS:DDA:SPC liposomes).

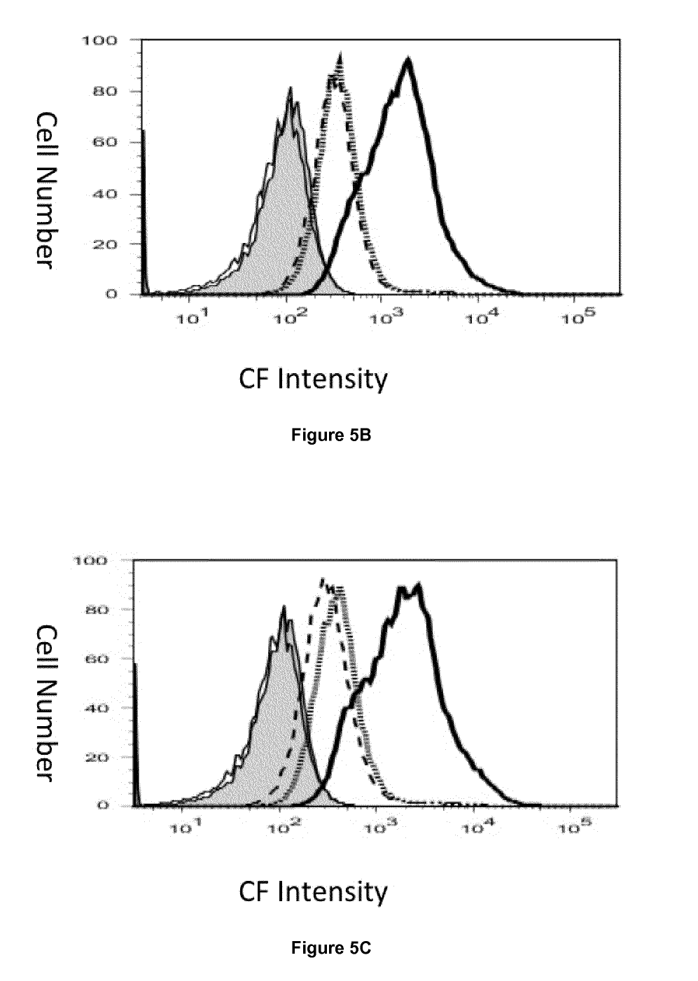

FIG. 6 shows CLSM images of lung epithelial cells incubated for 1 h with (A) Free CF, (B) CF-Loaded SPC liposomes, and (C) CF-Loaded TPGS:DDA:SPC liposomes.

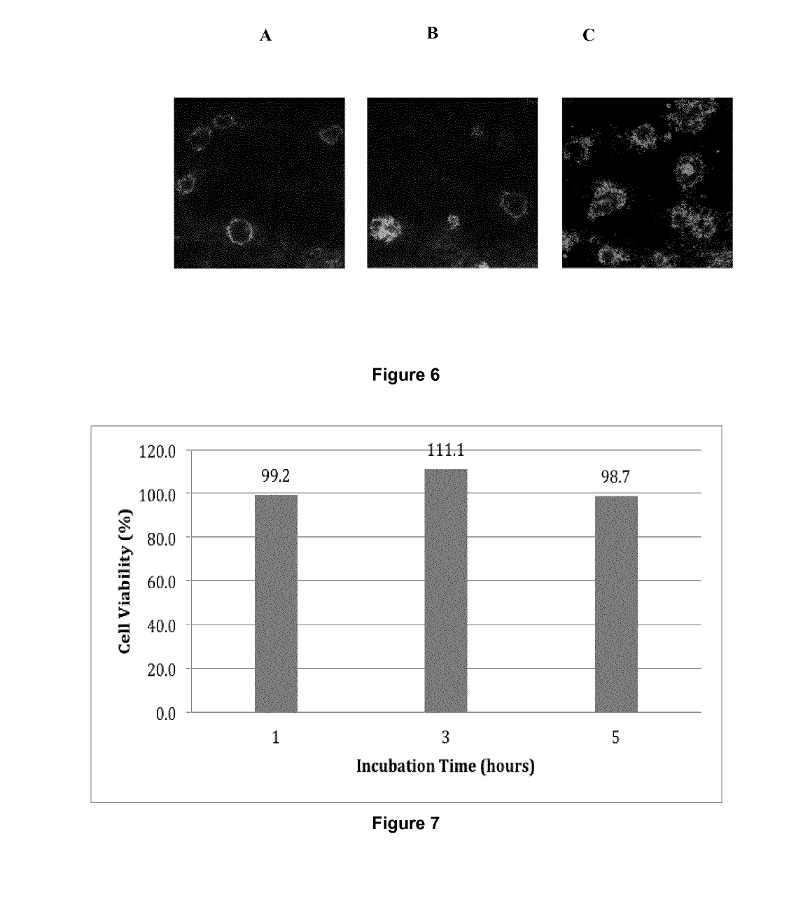

FIG. 7 shows quantitative cell viability measurement obtained by WST-1 assay to describe cell viability in the presence of TPGS:DDA:SPC liposomes by measuring the metabolic activity of viable cells with respect to the control after 1, 3 and 5 hours.

FIG. 8 shows DSC data for the phase transitions of hydrated TPGS:DDA:SPC compositions. TPGS was added at concentrations of 0.2, 0.6, 1.1, 2.1, 4.1, 6.1, 17.7, 30.1 and 39.3% by mass of the liposome components as indicated in the Figure.

### EXAMPLES

### General Materials and Methods

Materials

TPGS (high purity), DDA (>98% in purity), bovine serum albumin (BSA), Bradford's reagent, Coomasie Blue propidium iodide, and phosphotungstic acid Triton-X and sodium hydroxide were purchased from Sigma-Aldrich (UK), SPC (Lipoid S75) was purchased from Lipoid (Germany). Trehalose dihydrate (high purity, low endotoxin) was purchased from Ferro Pfanstiehl (USA). Chloroform and ethanol (analytical grade) was purchased from VWR (France). Tris(hydroxymethyl)methylamine was purchased from VWR (England). NuPAGE® MES SDS Running Buffer, nucleic acid sample loading buffer and Novex 4-12% Bis-Tris Gel (1.0 mm, 10 Well) were purchased from Invitrogen (UK). SimplyBlue™ SafeStain was purchased from Bio-Rad (UK). MEM medium, Earle's salts, fetal calf serum (FCS) and L-glutamine were purchased from Invitrogen (Paisley, UK). 24 and 96 well plates were from Sterilin (Newport, UK).

Liposome Manufacture

Liposomes were prepared based on the thin film hydration technique. Briefly, all lipid components and TPGS were dissolved in a chloroform/methanol (9:1) solvent system (1 ml). Solvent was removed with a rotary evaporator to form a lipid film. The lipid film was then hydrated with 2 ml tris buffer (1.21 mg/ml pH 7.4) containing 5% w/v trehalose. Samples were vortexed for 3 min and then transferred to a shaking water bath (57° C.) for 1 hour. The liposomal dispersion was then sonicated (3 mm diameter probe sonicator in) for 4 cycles of 1 min sonication and 30 s rest. The final dispersion was freeze dried for 24 hours (Advantage freeze-dryer, Virits, Warminster, US).

Liposome Size Determination

Samples (150 μl) were diluted with deionized water up to 4 ml. Samples were measured using a Zetasizer (Malvern Zetasizer, Malvern Instruments, UK) at 25° C. using 4 repeat measurements, the size was reported as the mean of the z-average values obtained. Freeze-dried samples were reconsituted to their initial volume with water prior to dilution and analysis as above.

Zeta Potential Determination

Samples (100 μl) were diluted with tris buffer (pH 7.4) up to 10 ml. Samples were measured using a Zetasizer (as before) at 25° C. taking the mean value of 10 repeats. The zeta potential parameter was then determined by using the mean of the results.

Glass Transition (Tg) Determination

Tg measurements were made on portions of the freeze dried products by moduated temperature differential scanning calorimetry (DSC). Samples were weighed in hermetic pans and analysed using a TA Q100 Differential Scanning calorimeter (Q100, TA Instruments, which had been calibrated for temperature, enthalpy and heat capacity) in modulated temperature mode using the following conditions: ramp from 0° C. to 200° C. at 2° C./min with a modulation of +/−0.4° C. every 40 seconds, 3 repeats were performed and the results are reported as the mean of these values with the standard deviation.

Water Content Determination

The water content in the freeze dried cakes was measured using thermogravimetric analysis (TGA Q500, TA instruments). Representative portions of the samples in aluminum pan were heated at 10° C./min from 25° C. to 200° C., (n=3) the results are reported as the mean of these values with the standard deviation.

### Example 1

### Method

Preparation of BSA-Loaded Liposomes

Liposome formulations which contained 1.0% TPGS, 16.6% DDA, and 82.4% SPC by mass composition of the liposomes were prepared based on the thin film hydration technique as described in the general method. Liposome formulations which contained 16.8% DDA and 83.2% SPC by mass liposome composition of were prepared in the same way. Sonication was used to obtain large unilamellar vesicles. Entrapment of BSA into the liposomes was performed using the dehydration-rehydration (DRV) method (Mayer et al, 1986, Gregoriadis et al, 2003). BSA was dissolved in tris buffer and 264 mM trehalose to give a concentration of 1 mg/ml BSA and mixed with the liposomes. Ultracentrifugation was used to separate free BSA. The liposomes entrapping BSA were freeze-dried (Advantage, VirTis, USA) for 36 h to obtain dry cakes.

Liposomal Characterization

The physicochemical characterizations include: The liposome size and zeta potential (Malvern Zetasizer, Malvern Instruments, UK), encapsulation efficiency (Spectrophotometer, Jenway-Genova, UK), water content (thermogravimetric analysis/TGA, TA instruments Q500), and thermal analysis (differential scanning calorimetry/DSC, TA instruments Q100). The analysis of BSA concentration was performed using Bradford's method. In vitro release experiments were performed using the dialysis method (300 000 Mw cut off) in PBS buffer (pH 7.4). The structural integrity of BSA before and after lyophilization was examined by sodium dodecyl sulphate-polyacrylamide gel electrophoresis (SDS-PAGE), and compared with native BSA. The gel (10 wells) was loaded with 10 μl of the sample and allowed to run for 2 h at 150 mV. The gel was stained with Coomassie blue to reveal the protein bands followed by destaining with deionized water.

Encapsulation Efficiency

The determination of BSA concentration for encapsulation efficiency (EE) used UV spectrophotometry after the samples were treated with Bradford's reagent to obtain a colored solution. The color is obtained upon the proportional binding of the dye Coomassie Blue G-250 to the protein amino acid. BSA was used as a standard instead of another protein standard to avoid technical error. The intensity of the color absorbance should be in linear correlation with the protein concentration. In order to validate the measurements, a series of BSA solutions in different concentrations used to construct a calibration curve i.e. 0; 1; 2; 4; 6; 8; and 10 μg/ml. The BSA concentrations in the liposome were expected to fall in this range of concentrations. The encapsulation efficiency was measured by determining the amount of the unencapsulated BSA and theoretical total loaded BSA. Unencapsulated BSA (free BSA) was separated by ultracentrifugation (Optima™ TLX ultracentrifuge; Bechman Coulter, USA) at 100,000 g for 25 min at 4° C. Supernatant (500 μl) was treated with 500 μl Bradford's reagent and incubated for at least 10 min. The absorbance was measured at 595 nm using a UV spectrophotometer (Jenway, Genova, UK). A linear calibration curve (r≧0.99) from standard solutions was used to determine the amount of BSA. Measurements were made in triplicate. The encapsulation efficiency of the liposomes was calculated from the ratio of unentrapped BSA and initial BSA loading using the following equation: EE=[(Ct−Cs)/Ct]×100%

Where Ct is the total concentration of BSA loaded and Cs is the concentration of BSA in the supernatant (untrapped BSA) after liposomes were separated from the unentrapped BSA by ultracentrifugation.

Drug Release Study

in vitro release experiments of BSA from liposomes were performed at 37°±0.5° C. using the dialysis method under constant shaking (60 rpm). A 10 ml of phosphat buffered saline (PBS) pH 7.4 was used to test for the drug release for 48 h. Dialysis bags (Spectra/Por Biotech Cellulose Ester Dialysis Membrane MWCO: 300,000 Spectrum Ls Rancho Dominguez, Calif., USA) containing 500 μl liposomes were introduced into the system. All of the release media was withdrawn and replaced with the same volume of fresh medium at designated time points to maintain sink conditions. The sampled medium (1.0 ml out of 10 ml) was mixed with Bradford's reagent and the absorbance was measured as described in the method for determination of encapsulation efficiency. The concentration of the BSA released from the liposomes was calculated from the obtained linear calibration curve (r≧0.99) as previously described in the method for determination of encapsulation efficiency.

Statistical Analysis

Statistical analysis of the data was carried out by the unpaired t test, Welch corrected (GraphPad Software, Inc., USA).

Results

TABLE 1
Effect of BSA encapsulation (0.5 mg/ml) on liposome size and zeta potential of
prepared liposomes before and after freeze-drying (FD). “TPGS:DDA:SPC
liposomes” refers to liposomes containing 1.0% TPGS, 16.6% DDA and
82.4% SPC (corresponding to Formulation 11 in Example 3). “DDA:SPC
liposomes” refers to liposomes containing 16.8% DDA and 83.2% SPC
by mass (corresponding to Formulation 3E in Example 3). (n = 4, ± represents SD).

| | Liposome Size (nm) | | Zeta Potential (mV) | |
|---|---|---|---|---|
| | DDA:SPC | TPGS:DDA:SPC | DDA:SPC | TPGS:DDA:SPC |
| Empty Liposomes | 88.8 ± 0.6 | 97.5 ± 0.3 | 62.6 ± 0.3 | 41.3 ± 1.0 |
| Before freeze drying | 184.8 ± 22.9 | 193.6 ± 7.7 | 45.3 ± 5.0* | 33.0 ± 3.2 |
| After freeze drying | 267.4 ± 19.5 | 248.3 ± 37.5 | 55.8 ± 3.2* | 47.4 ± 4.9 |

TABLE 2
Encapsulation efficiency of liposomes determined before drying and
the dry product characteristics of liposomes prepared by mixing the
liposome drug delivery system with 0.5 mg/ml BSA in the presence
of 264 mM trehalose to form the liposome therapeutic agent delivery
complex (n = 3, ± represents SD). *p < 0.05 as
compared to those without TPGS).

| Formulation | BSA Encapsulation | | Water Content |
|---|---|---|---|
| % composition | Efficiency (%) | Tg (° C.) | (%) |
| DDA:SPC 16.8:83.2 | 93.4 ± 1.7 | 74.9 ± 3.3 | 2.9 ± 0.4 |
| TPGS:DDA:SPC | 96.6 ± 0.8 | 66.2 ± 3.1* | 4.1 ± 0.4* |
| (1.0:16.6:82.4) | | | |

The formulation without TPGS experienced significant increased in size (p<0.05) while the incorporation of TPGS in formulation successfully maintained the size after freeze drying (Table 1). This demonstrates the significant role that TPGS has in maintaining the structure of the membrane. However, the zeta potential values were significantly different before and after freeze-drying for both formulations (p<0.05). This can be explained by a reduction in the amount of negatively charged BSA on the surface of the liposomes as the protein was transferred within liposomes during the rehydration process. Furthermore, the increase in BSA loading has also resulted in an increase in liposome size as well as the zeta potential.

There were no significant differences in encapsulation efficiency for either formulation (Table 2). Nevertheless, the formulation containing TPGS exhibited a slightly better efficiency than those without TPGS. Again, the influence of TPGS on membrane fluidity might enhance transmembrane transport of BSA beside the different concentration of protectant across the lipid bilayer which also acts as a driving force.

For drug release characterization, the free BSA showed a cumulative release of 46.6±2.6% at 4 h, showing that there was no effect of BSA binding on the dialysis membrane which might influence the drug release profile (FIG. 1). The two formulations showed different release profiles between when comparing samples of freshly prepared with liposomes with those that had been freeze-dried. Freshly prepared BSA loaded liposomes showed releases of 26.6±0.3% (DDA-SPC) and 33.6±1.1% (TPGS:DDA:SPC) for the first 4h. This implies a burst release effect due to the presence of some BSA on the surface of the liposomes. Furthermore, BSA-loaded liposomes exhibited gradual increase of releases with total cumulative release of 72.4±0.5% (DDA-SPC) and 78.1±1.5% (TPGS:DDA:SPC) at 24 h and 85.5±2.2% and 84.9±3.6% at 48 h for DDA-SPC and TPGS:DDA:SPC respectively. The release patterns were similar for both freeze-dried formulations which is a more controlled release pattern. This can be explained as the BSA was encapsulated inside the liposomes during the rehydration process and therefore, no burst effect of BSA being released from the surface of liposomes was observed. The controlled release of the freeze-dried liposomes showed that only 22.6±1.0% (DDA:SPC) and 31.6±0.7% (TPGS:DDA:SPC) release after 24 h. The release values were still <50% where only 25.6±3.6% (DDA:SPC) and 40.3±1.6% (TPGS:DDA:SPC) of the protein had been released after 48 h.

SDS-PAGE analysis of the BSA-loaded liposomes before and after lyophilisation revealed no signs of damage to the BSA (FIG. 2) indicating that the structural integrity of BSA was not significantly altered by either the entrapment or drying procedures. From the same figure, we could also see tailing shaped of BSA from the formulations, which can be explained as the effect of lipids from liposomes that were not separated completely.

### Example 2

To investigate the ability of the TPGS:DDA:SPC liposomes of Example 1 to cross nasal mucosa and penetrate into bovine nasal tissue fresh bovine nasal tissues were collected from freshly slaughtered animals. The mucosal tissue was removed and preserved in Krebs Bicarbonate Ringer (KBR) buffer pH 7.4. Liposome penetration studies were performed immediately to maintain the viability of the cells tissue being used. A Franz type permeation cell was used in this study; the recipient compartment filled with KBR buffer and stirred constantly. The temperature of the Franz cell was maintained at 37° C. with jacketed water flow. A 1.5 cm² tissue section was inserted between the recipient and donor compartments and 0.5 ml of reconstituted liposomal dispersion or free carboxyfluorescein (CF) solutions containing an equivalent amount of CF were loaded into the donor compartment, and left for 1 hour incubation. The tissues were then washed with KBR buffer and frozen in optimal cutting temperature compound at −80° C. for cryosection. Samples of 10 μm thickness were obtained using a cryostat (MICROM HM 550). Propidium iodide (PI) solution was used to stain the cell nuclei and cryosectioned tissues were mounted in glycerol containing 1,4-phenylenediamine (0.1 g/l). Penetration was observed using confocal laser scanning microscopy (CLSM). Images of nasal tissues were acquired using an excitation wavelength 488 nm and captured with Nikon C1s (Nikon PlanApo 60x, 1.4-NA water immersion lens.

CLSM images show that free CF was completely washed out and did not even remain at the superficial epithelial layer (FIG. 3). Conversely, the majority of the both the SPC liposomes and the TPGS:DDA:SPC liposomes permeated through the superficial epithelium while the TPGS:DDA:SPC liposomes showed the highest concentration (brightness) and distribution at the basal epithelium.

Cell Uptake of Liposomal Formulations:

Cells from the human lung epithelial cell line L-132 were cultured in MEM medium containing Earle's salts and L-glutamine and supplemented with 10% fetal calf serum (FCS), under standard conditions at 37° C. with 5% CO₂ and 95% air. To validate the fluorimetric analysis, a standard curve was created during each assay. To perform the fluorometric assay cells were seeded at 25×10⁴ cells/well onto 24-well plates (total volume was 1 ml/well). Twenty four hours later, the culture medium was removed. The formulations were added to the cells in 1:100 dilution in MEM medium (10 μl of liposomes suspension+990 μl medium) and incubated for 0.5-2 h at 37° C. Following incubation cells were washed two times with cold PBS to end the uptake experiment. Then the washed cells were lysed with 0.2% Triton X-100 in 0.2 N NaOH (1.0 ml/well) for 0.5-1 hour at room temperature. Aliquots from each well were transferred into 96-well plates. The cell fluorescence intensity was determined using a microplate fluorometer at an excitation wavelength of 485 nm and emission wavelength of 520 nm and Gain level set to 800 in the software (FIG. 4). For flow cytometry a similar procedure as fluorometric assay was applied to the samples. However, cells were seeded at 50×10⁴ cells/well onto 24-well plates (total volume was 1 ml/well) and following incubation, cells were washed one time with cold PBS to end the uptake experiment. Then the washed cells were detached from the plate by adding trypsin (200 μl/well) and incubated for 3 minutes at 37° C. 1 ml of PBS was added to each well and mixed gently before transferral to an Eppendorf tube. The trypsin was removed from the by centrifugation at 1500 rpm, at 4° C. for 5 min. The precipitated cells were resuspended in 400 μl PBS and gently mixed to avoid cell aggregation. The cells were kept on ice and analysized by flow cytometry using a FACscan flow cytometer for cell-associated CF fluorescence. The mean fluorescence intensity of cells was determined for each sample (FIG. 5). The cells were seeded at 2×10⁵ cells/well onto a 4-well culture slide. Twenty-four hours later, the culture medium was replaced with Free CF and the liposome formulations. After incubation for 1 h at 37° C., the cells were washed twice with cold PBS (pH 7.4) and then fixed with 4% paraformaldehyde. The fixed cells were observed using CLSM (FIG. 6). We found that the amount of CF incorporated into cells were increased in the order of free CF<CF-loaded SPC liposomes <CF-loaded TPGS:DDA:SPC liposomes. CF-loaded TPGS:DDA:SPC liposomes were superior in delivering CF into the epithelial cells compared to those CF-loaded conventional SPC liposomes.

Cell viability was measured using a colorimetric assay for 96-well plates with WST-1 reagent (2-(4-iodophenyl)-3-(4-nitrophenyl)-5-(2,4-disulfophenyl)-2H-tetrazolium monosodium salt). Prepared plate contained blanks and formulation with three different incubation times (four replicates each). Cells were added to the plate in a concentration of 25×10³ cells/well (200 μl MEM medium with 10% FCS) and cultivated for 24 h. After seeding, the TPGS:DDA:SPC based liposomes were diluted with the same medium to give a 1:10 dilution (100 μl of liposomes suspension+900 μl medium). A 200 μl aliquot of the diluted formulation was then added to the cells and incubated for 1-5 h at 37° C. Following incubation, the formulation was removed and replaced with 100 μl/well medium without FCS. 10 μL of WST-1 were then added and cells were incubated for an additional 2 h with this reagent. Cell viability was measured at 450 nm in a microplate reader (Bio-Tek Instruments EL808 Microplate Reader). Cytotoxicity of the formulation (compounds) was expressed as percentage cell viability (measured as WST-1 reduction by viable cells) compared to controls (FIG. 7). Cells demonstrated good viability across the 5 hours of the study, indicating that the TPGS:DDA:SPC liposomes are not toxic.

Conclusion from Example 1 and Example 2

Physicochemical characterization; liposome size, zeta potential, encapsulation efficiency, drug release, protein stability, water content and glass transition temperature of the TPGS:DDA:SPC liposomes have shown positive results. Mucosal penetration and cell uptake data have also shown that TPGS:DDA:SPC liposomes are suitable for use as immunological adjuvant/vaccine delivery systems. Cell viability data indicate that the TPGS:DDA:SPC liposomes are non-toxic to the cells studied. These studies were performed with liposomes that had been reconsituted from freeze-dried products indicating that the TPGS:DDA:SPC liposomes are able to perform effectively after the freeze-drying and reconstitution process.

### Example 3

### Optimising the Liposome Formulation

Effect of SPC Concentration on Stability of DDA:SPC Liposomes to Freeze-Drying

Liposomes were manufactured and sized as described in the general method with the following modification, compositions were as described in Table 1.

Results

Table 1 shows the effect of increasing SPC concentration on the size of DDA Liposomes.

| | | | | | Size before | Size after |
|---|---|---|---|---|---|---|
| | DDA | SPC | Molar ratio | % Composition | freeze drying | freeze drying |
| | (mg/ml) | (mg/ml) | DDA:SPC | DDA:SPC | (nm) | (nm) |
| Formulation | 2.5 | — | 1:0 | 100:0 | 107.8 ± 0.14 | 1673.8 ± 358.04 |
| 1A | | | | | | |
| Formulation | 2.5 | 2.325 | 4:3 | 51.8:48.2 | 121.6 ± 11.2 | 1067.0 ± 38.7 |
| 2D | | | | | | |
| Formulation | 2.5 | 12.4 | 4:16.3 | 16.8:83.2 | 121.78 ± 88.8 ± 0.6 | 102.2 ± 0.3 |
| 3E | | | | | | |

Table 1 shows that increasing SPC concentration increases the stability of the liposomes to freeze drying. However, these liposomes do not contain TPGS necessary for adequate penetration of biological tissue.

Effect of TPGS Concentration on Stability of DDA Liposomes after Freeze Drying

Liposomes were manufactured and sized as described in the general method. Formulations contained ratios of DDA and TPGS as described in Table 2 below.

Results

TABLE 2
Compositions and properties of liposomes containing varying concentrations of TPGS.

| | | | | | Liposome | Liposome |
|---|---|---|---|---|---|---|
| Liposome | DDA | TPGS | DDA:TPGS | % Composition | size before | size after |
| Example | (mg/ml) | (mg/ml) | Molar ratio | DDA:TPGS | freeze drying | freeze drying |
| 1A | 2.5 | — | 1:0 | 100:0 | 107.80 ± 0.14 | 1673.80 ± 358 |
| 4B | 2.5 | 0.025 | 240:1 | 99:1 | 100.63 ± 4.35 | 1057.80 ± 115 |
| 5C | 2.5 | 0.05 | 120:1 | 98:2 | 103.57 ± 23.0 | 552.30 ± 13.0 |

Table 2 shows that DDA:TPGS alone can form liposomes that are of an acceptable size range when freshly prepared but can not withstand the freeze-drying process as evidenced by the drastic increase in liposome size.

Effect of Storage on Stability of Dispersions Containing DDA and TPGS Alone

Freshly prepared portions of the formulations described in Table 2 were stored at 4° C. Measurements were made on days 1, 3, 5, 7 and 9 using the sizing method as previously.

Results

TABLE 3
Liposome size stabilty during 9 days storage as liquid dispersions at 4° C.

| | Day 1 Size | Day 3 Size | Day 5 Size | Day 7 Size | Day 9 Size |
|---|---|---|---|---|---|
| | (nm) | (nm) | (nm) | (nm) | (nm) |
| Formulation | 107.80 ± 0.14 | 208.60 ± 4.51 | 292.13 ± 6.54 | 391.20 ± 26.50 | 434.50 ± 18.31 |
| 1A | | | | | |
| Formulation | 100.63 ± 4.35 | 112.63 ± 9.60 | 148.40 ± 3.94 | 129.97 ± 0.64 | 153.40 ± 20.37 |
| 4B | | | | | |
| Formulation | 103.57 ± 22.97 | 185.73 ± 4.83 | 311.83 ± 7.09 | 451.83 ± 5.71 | 657.07 ± 12.22 |
| 5C | | | | | |

Table 3 shows that Formulation 4B, containing 1% TPGS performs better than both the Formulation with no TPGS and the example with 2% TPGS demonstrating that there is an optimum level of TPGS necessary for DDA:TPGS liposome stability.

Effect of TPGS Concentration on the Size of DDA:SPC (80:329) Liposomes.

Liposomes were manufactured and sized as described in the general method with the following modification; the buffer contained 10% trehalose. Formulations contained ratios of DDA, SPC and TPGS between 0.5 and 7.7% total liposome content as described in Table 4.

Results

TABLE 4
The effect of TPGS concentration on the size of freshly prepared
and freeze-dried DDA:SPC (molar ratio 80:329) Liposomes.

| | | | | | | Size before | Size After |
|---|---|---|---|---|---|---|---|
| | DDA | SPC | TPGS | Molar ratio | % Composition | freeze drying | freeze drying |
| | (mg/ml) | (mg/ml) | (mg/ml) | DDA:SPC:TPGS | DDA:SPC:TPGS | (nm) | (nm) |
| 6 | 2.5 | 12.4 | 0.075 | 80:329:1 | 16.7:82.8:0.5 | 96.6 ± 0.80 | 111.9 ± 0.91 |
| 7I (same | 2.5 | 12.4 | 0.15 | 80:329:2 | 16.6:82.4:1.0 | 97.5 ±0.32 | 113.1 ± 0.62 |
| ratio as | | | | | | | |
| 11) | | | | | | | |
| 8 H | 2.5 | 12.4 | 0.30 | 80:329:4 | 16.5:81.6:2.0 | 98.2 ± 0.52 | 118.3 ± 0.29 |
| 9 G | 2.5 | 12.4 | 1.2 | 80:329:8 | 15.5:76.8:7.7 | 91.2 ±1.45 | 136.9 ± 1.0 |

Table 4 shows that for the DDA:SPC liposomes prepared with the molar ratio of 80:329 for DDA and SPC respectively, the highest concentration of TPGS shows the smallest liposomal size when the liposomes are freshly prepared but the largest increase once they have been freeze-dried.

Effect of TPGS Concentration on the Size of DDA:SPC (Molar Ratio 39:31) Liposomes.

Liposomes were manufactured and sized as described in the general method with the following modification; the buffer contained 10% trehalose. Formulations contained ratios of DDA, SPC and TPGS as described in Table 5. Table 5 shows the effect of TPGS concentration on the size of freshly prepared and freeze-dried DDA:SPC (molar ratio 39:31) liposomes.

Results

TABLE 5

| | | | | | | Size Before | Size After |
|---|---|---|---|---|---|---|---|
| | DDA | SPC | TPGS | Molar ratio | % Composition | freeze drying | freeze drying |
| | (mg/ml) | (mg/ml) | (mg/ml) | DDA:SPC:TPGS | DDA:SPC:TPGS | (nm) | (nm) |
| Formulation | 2.5 | 2.325 | — | 39:31:0 | 51.8:48.2:0 | 121.6 ± 11.2 | 1067.0 ± 38.7 |
| 2D | | | | | | | |
| Formulation | 2.5 | 2.325 | 0.15 | 39:31:1 | 50.3:46.7:3.0 | 146.2 ± 14.3 | 256.7 ± 4.5 |
| 10F | | | | | | | |

Table 5 shows that DDA:SPC liposomes are not stable at the ratio 39:31 even when TPGS is included at the concentration shown in Formulation 10F.

The Effect of BSA Concentration on Liposome Size.

The liposomes were manufactured and sized as described in the general method with the following modifications; the DDA, SPC and TPGS concentrations were as described in Table 6; the buffer contained 10% trehalose. Three different BSA concentrations were prepared as described in Table 6 and mixed with the prepared liposomes as described in Example 1.

TABLE 6

| | Concentrations used to make | | | | |
|---|---|---|---|---|---|
| | initial liposomal dispersions | | | | |
| | (mgml) | | | % Ratio | BSA |
| Formulation | DDA | SPC | TPGS | DDA:SPC:TPGS | (mg/ml) |
| 3E | 2.5 | 12.4- | — | 16.8:83.2 | 0 |
| 11 | 2.5 | 12.4- | 0.15 | 16.6:82.4:1.0 | 0 |
| 12 | 2.5 | 12.4- | — | 16.8:83.2 | 0.25 |
| 13 | 2.5 | 12.4- | 0.15 | 16.6:82.4:1.0 | 0.25 |
| 14 | 2.5 | 12.4- | — | 16.8:83.2 | 0.5 |
| 15 | 2.5 | 12.4- | 0.15 | 16.6:82.4:1.0 | 0.5 |
| 16 | 2.5 | 12.4- | — | 16.8:83.2 | 1.0 |
| 17 | 2.5 | 12.4- | 0.15 | 16.6:82.4:1.0 | 1.0 |

Results:

TABLE 7
Effect of BSA concentration on liposome size

| | % Ratio | BSA | Liposome size |
|---|---|---|---|
| Formulation | DDA:SPC:TPGS | (mg/ml) | (nm) |
| 3E | 16.8:83.2 | 0 | 113 ± 0.8 |
| 11 | 16.6:82.4:1.0 | 0 | 106.17 ± 0.4 |
| 12 | 16.8:83.2 | 0.25 | 124.57 ± 1.36 |
| 13 | 16.6:82.4:1.0 | 0.25 | 130.33 ± 1.88 |
| 14 | 16.8:83.2 | 0.5 | 172.7 ± 2.4 |
| 15 | 16.6:82.4:1.0 | 0.5 | 191.23 ± 6.93 |
| 16 | 16.8:83.2 | 1.0 | 258.7 ± 7.3 |
| 17 | 16.6:82.4:1.0 | 1.0 | 282.13 ± 11.85 |

The results in Table 7 show that adding BSA to the liposomes increases the liposomal size for both liposomes with and without TPGS. The size of liposomes formulated with drug, therapeutic agent or antigen will vary dependent on the size, nature and loading of the therapeutic agent.

Conclusions from Example 3:

We found that the DDA liposomes are not stable if DDA is the major component of the lipid composition even if TPGS is included. We have found that SPC stabilises DDA liposomes if it is the major component of the lipid composition. Liposomes composed of DDA and TPGS alone are not stable to freeze drying, although storage stability data obtained indicate that there is an optimum concentration of TPGS required for stability of DDA:TPGS liposomes and that if this level is exceeded then these liposomes become very unstable upon storage. When TPGS is incorporated into DDA:SPC liposomes that are formed with lipid molar ratios of DDA:SPC 80:329 respectively, then higher concentrations of TPGS give the smallest size when freshly prepared but are the least stable to the freeze drying process, indicating that lower TPGS concentrations are preferable for the formation of stable TPGS:DDA:SPC liposomes. When TPGS is incorporated into DDA:SPC liposomes that are formed with lipid molar ratios of DDA:SPC 39:31 respectively then addition of TPGS cannot stabilise these liposomes to freeze-drying. Addition of therapeutic agent to the liposomal delivery system will increase the liposome size, the increase will vary according to the size, nature and loading of the therapeutic agent.

### Example 4

### Phase Behaviour of Rehydrated Lyophilized Liposome Compositions

Materials

Materials were from the same source and purity as the previous examples.

Sample Preparation

SPC, DDA, and TPGS were dissolved and mixed in chloroform/methanol (9:1). DDA and SPC concentrations were kept constant at 2.5 mg/ml and 12.4 mg/ml respectively (as per the previous examples) while TPGS was added at concentrations of 0.2, 0.6, 1.1, 2.1, 4.1, 6.1, 17.7, 30.1 and 39.3% by mass of the liposome components. Solvent was removed under nitrogen before placing under vacuum overnight. The lipid film was then hydrated with pre-heated (60° C.) 10 mM tris buffer (pH 7.4). Samples were stirred for 30 min at 60° C. The liposome dispersion was reduced in size by sonication in pulsative mode for 2 minutes (Fisher Scientific, Sonic Dismembrator, Model 120, Pittsburgh, Pa., USA). Samples were freeze-dried for 40 h (Advantage, VirTis, USA). Accurately weighed 2.5 mg samples of the freeze-dried lipid compositions were rehydrated with 15 μl distilled water directly in the hermetic aluminium DSC pan immediately prior to phase transition temperature determination.

Phase Transition Determination

DSC (Q-100, TA Instruments, USA) was used to determine the phase transition temperature (Tm) of the lipids. The rehydrated samples were incubated at 80° C. for 30 minutes in the DSC then scanned from 30° C. to 60° C. at 1° C./min. All samples were measured in three replicates. Data were analyzed using Universal Analysis Software. Tm was determined from the endotherm peak minimum for the thermotropic phase transition of membrane lipids in the presence of water.

Results

Phase separation in liposomal membrane may have major disadvantage for their application as delivery systems. A phase-separated component from the rest of the membrane will allow leakage of the encapsulated drug or even make the liposome collapse. Therefore, the effect of TPGS on the phase behaviour (i.e. miscibility and phase separation) of dehydrated phospholipids mixtures was investigated. The DSC data from the binary lipid mixture (DDA and SPC alone, labelled “0%” on FIG. 8) showed one endothermic peak at 60° C. (labelled “Upper” on FIG. 8), indicating that no phase separation had occurred during the dehydration process in this composition and therefore, indicating the miscibility of the two lipids. Addition of TPGS to the binary mixture showed phase separation of TPGS beginning to occur at TPGS concentrations ≧1% where a lower peak attributable to the TPGS became apparent (labelled “lower” in FIG. 8). The peak attributed to TPGS became more obvious as the concentration of TPGS increased. Only TPGS at lower concentration i.e. 0.1% and 0.3% did not show any phase separation in the lipid mixtures. When considering the upper endothermic peak, it was observed that at concentrations ≧4.1% TPGS the upper peak had begun to split, indicating phase separation of DDA and SPC.

Conclusion from Example 4

The phase separation observations described in the results section indicate that the stability of the TPGS:DDA:SPC compositions decreases at TPGS concentrations 4%.

### REFERENCES

Brandt L, Elhay M, Rosenkrands I, Lindblad E B, Andersen P. ESAT-6 subunit vaccination against Mycobacterium tuberculosis. Infection and Immunity 2000 February; 68(2):791-5).
Fahy, E, Subramaniam, S, Murphy, R C, Nishijima, M, Raetz, C R H, Shimizu, T, Spener, F, van Meer, G, Wakelam, M J O, and Dennis, E A Update of the LIPID MAPS comprehensive classification system for lipids. Journal of Lipid Research 2009 April; 50(Supplement): S9-S14.
Gregoriadis G, Bacon A, Caparros-Wanderley W, McCormack B. Plasmid DNA vaccines: Entrapment into liposomes by dehydration-rehydration. Liposomes, Pt A 2003; 367:70-80.
Hincha D K. Effects of alpha-tocopherol (vitamin E) on the stability and lipid dynamics of model membranes mimicking the lipid composition of plant chloroplast membranes. Febs Letters 2008 Oct. 29; 582(25-26):3687-92].
Holten-Andersen L, Doherty T M, Korsholm K S, Andersen P. Combination of the cationic surfactant dimethyl dioctadecyl ammonium bromide and synthetic mycobacterial cord factor as an efficient adjuvant for tuberculosis subunit vaccines. Infection and Immunity 2004 March; 72(3):1608-1
Maherani, B, Arab-Tehrany, E, Mozafari, M R, Gaiani, C, Linder, M Liposomes: A Review of Manufacturing Techniques and Targeting Strategies. Current Nanoscience 2011 June; 7: 436-452
Mayer L D, Bally M B, Hope M J, Cullis P R. Techniques for Encapsulating Bioactive Agents Into Liposomes. Chemistry and Physics of Lipids 1986 June; 40(2-4):333-45.
Mohammed A R, Bramwell V W, Coombes A G A, Perrie Y. Lyophilisation and sterilisation of liposomal vaccines to produce stable and sterile products. Methods 2006 September; 40(1):30-8
Somavarapul S, Pandit S, Gradassi G, Bandera A, Ravichandran E, Alpar O H. Effect of vitamin E TPGS on immune response to nasally delivered diphtheria toxoid loaded poly(caprolactone) microparticles. International Journal of Pharmaceutics 2005 Jul. 25; 298(2):344-7.
Wang Y Y, Lai S K, Suk J S, Pace A, Cone R, Hanes J. Addressing the PEG Mucoadhesivity Paradox to Engineer Nanoparticles that “Slip” through the Human Mucus Barrier Angew. Chem. Int. Ed 2008 47, 9726-9729

1. A liposomal delivery system comprising discrete liposomes, the system comprising
a minor lipid component dimethyldioctadecylammonium (DDA);
a major lipid component or components selected from the group consisting of glycerolipid, glycerophospholipid, sphingolipid, and a combination thereof which is capable of forming a stable lipid bilayer with the minor lipid component dimethyldioctadecylammonium (DDA); and
a tocopherol polyethylene glycol (PEG) ester derivative;
wherein the liposomal delivery system does not comprise the lipid cholesterol.

2. The liposomal delivery system of claim 1 wherein the major lipid component is present in the liposomal delivery complex in an amount greater than the minor lipid component.

3. The liposomal delivery system of claim 1 wherein the amount of the major lipid component in the liposomal delivery complex is greater than the amount of the minor lipid component DDA which is greater than the amount of the tocopherol polyethylene glycol (PEG) ester derivative.

4. The liposomal delivery system of claim 1 comprising less than approximately 50% by weight, based on the total weight of the system, of the minor lipid component dimethyldioctadecylammonium (DDA); approximately 50% or more, by weight based on the total weight of the system, of the major lipid component or components; and less than approximately 10% by weight, based on the total weight of the system, of a tocopherol polyethylene glycol (PEG) ester derivative.

5. The liposomal delivery system of claim 1 comprising less than approximately 20% by weight, based on the total weight of the system, of the minor lipid component dimethyldioctadecylammonium (DDA); approximately 80% or more, by weight based on the total weight of the system, of the major lipid component or components; and less than approximately 10% by weight, based on the total weight of the system, of a tocopherol polyethylene glycol (PEG) ester derivative.

6. The liposomal delivery system of claim 1 comprising from 15% to 20% by weight based on the total weight of the system of the minor lipid component dimethyldioctadecylammonium (DDA), from 80% to 85% by weight based on the total weight of the system of the major lipid component or components, and from 0.1% to 10% by weight based on the total weight of the system of the tocopherol polyethylene glycol (PEG) ester derivative.

7. The liposomal delivery system according to claim 1 consisting of the minor lipid component dimethyldioctadecylammonium (DDA); the major lipid component or components selected from the group consisting of glycerolipid, glycerophospholipid, sphingolipid, and a combination thereof; and the tocopherol polyethylene glycol (PEG) ester derivative.

8. The liposomal delivery system of claim 1, wherein the minor lipid component is dimethyldioctadecylammonium-bromide, dimethyldioctadecylammonium-chloride, dimethyldioctadecylammonium-phosphate and/or dimethyldioctadecylammonium-acetate.

9. (canceled)

10. The liposomal delivery system of claim 1 wherein the major lipid component is selected from the group consisting of diacylglycerophosphocholines, glycerophosphates, glycerophosphoethanolamines, glycerophosphoglycerols, glycerophosphoglycero-phosphoglycerols, and glycerophosphoserines.

11. The liposomal delivery system of claim 1 wherein the major lipid component is a glycerophosphocholine or phosphatidylcholine.

12. (canceled)

13. The liposomal delivery system of claim 1 wherein the tocopherol PEG ester derivative is present less than approximately 9% by weight based on the total weight of the system.

14. (canceled)

15. The liposomal delivery system of claim 1 wherein the tocopherol PEG ester derivative is selected from the group consisting of tocopherol sebacate polyethylene glycol, tocopherol dodecanodioate polyethylene glycol, tocopherol suberate polyethylene glycol, tocopherol azelaate polyethylene glycol, tocopherol citraconate polyethylene glycol, tocopherol methylcitraconate polyethylene glycol, tocopherol itaconate polyethylene glycol, tocopherol maleate polyethylene glycol, tocopherol glutarate polyethylene glycol, tocopherol glutaconate polyethylene glycol, and tocopherol phthalate polyethylene glycol.

16. The liposomal delivery system of claim 1 wherein the tocopherol PEG ester derivative is a delta tocopherol or alpha tocopherol PEG derivative.

17. (canceled)

18. The liposomal delivery system of claim 1 comprising the major lipid component phosphatidylcholine; the minor lipid component dimethyldioctadecylammonium (DDA); and the tocopherol PEG ester derivative D-alpha tocopherol polyethylene glycol succinate (TPGS).

19. (canceled)

20. (canceled)

21. (canceled)

22. (canceled)

23. The liposomal delivery system of claim 1 adapted for mucosal adminstration.

24. A bioactive agent liposomal delivery complex comprising the liposomal delivery system of claim 1 and a bioactive agent selected from the group consisting of vaccines, immunogenic compositions, proteins, nucleic acids, drugs/medicaments, therapeutic agents, imaging agents, and diagnostic agents.

25. (canceled)

26. (canceled)

27. (canceled)

28. A method for the delivery of a bioactive agent to a subject using the liposomal delivery system of claim 1 wherein the bioactive agent or agent is adsorbed to or encapsulated within the liposomal delivery system to form a bioactive agent liposomal delivery complex and the method comprises adminstering the bioactive agent liposomal delivery complex or the pharmaceutical composition to a subject in need thereof.

29. A liposomal delivery system comprising discrete liposomes, the system comprising
less than approximately 50% by weight based on the total weight of the system, of a minor lipid component dimethyldioctadecylammonium (DDA);
more than approximately 50% by weight based on the total weight of the system, of a major lipid component or components selected from the group consisting of glycerolipid, glycerophospholipid, sphingolipid, and a combination thereof which is capable of forming a stable lipid bilayer with the minor lipid component dimethyldioctadecylammonium (DDA); and
less than approximately 10%, by weight based on the total weight of the system, of a tocopherol polyethylene glycol (PEG) ester derivative.

30. The liposomal delivery system of claim 18 comprising less than approximately 20% by weight, based on the total weight of the system, of the minor lipid component dimethyldioctadecylammonium (DDA); approximately 80% or more, by weight based on the total weight of the system, of the major lipid component or components; and less than approximately 10% by weight, based on the total weight of the system, of a tocopherol polyethylene glycol (PEG) ester derivative.

31. The liposomal delivery system of claim 18 consisting of the minor lipid component dimethyldioctadecylammonium (DDA); the major lipid component or components selected the group consisting of glycerolipid, glycerophospholipid, sphingolipid, and a combination thereof; and the tocopherol polyethylene glycol (PEG) ester derivative.
